(19) United States
(12) Patent Application Publication
(10) Pub. No.: US 2026/0325411 A1
(43) Pub. Date: Oct. 22, 2026

(54) REUSABLE SILICONE GEL COSMETIC PATCH

(71) Applicant: Shenzhen Shengyou Technology Co., Ltd, Shenzhen (CN)

(72) Inventor: Shaojun Zhang, Shenzhen (CN)

(73) Assignee: Shenzhen Shengyou Technology Co., Ltd, Shenzhen (CN)

(21) Appl. No.: 19/227,545

(22) Filed: Jun. 4, 2025

(30) Foreign Application Priority Data
Apr. 18, 2025 (CN) 202520735963.X

(51) Int. Cl. A61K 8/02; A61K 8/25

(52) U.S. Cl. CPC A61K 8/0233; A61K 8/25

(57) ABSTRACT

Disclosed is a reusable silicone gel cosmetic patch, including a silicone base layer, a textile fabric layer, and a silicone gel layer, where the silicone base layer, the textile fabric layer, and the silicone gel layer are integrally formed, and the textile fabric layer is sandwiched between the silicone base layer and the silicone gel layer. The silicone base layer, the textile fabric layer, and the silicone gel layer are laminated and fixed together in sequence, and the textile fabric layer is sandwiched between the silicone base layer and the silicone gel layer. Since the textile fabric layer can withstand high temperatures, the cosmetic patch can be sterilized by high temperatures or boiling, making it suitable for repeated use. Due to the traction of the textile fabric layer, the cosmetic patch can be repeatedly applied and removed without deformation, thereby effectively solving the problem of resource waste.

### TECHNICAL FIELD

The present disclosure relates to the technical field of cosmetic products, and in particular to a reusable silicone gel cosmetic patch.

### BACKGROUND

A silicone gel cosmetic patch is a product specially designed for skincare and cosmetic purposes. It is made of silicone gel materials and has various properties beneficial to the skin. Currently, the silicone gel cosmetic patch is usually applied to specific areas of the skin after the use of cosmetic products such as essence and moisturizing lotion, with functions of locking in skin moisture, softening a stratum corneum, and improving the absorption of skincare products such as essence and moisturizing lotion. Therefore, it is an auxiliary product that helps improve the beauty effect.

In the prior art, the silicone gel cosmetic patch is usually made of plastic materials such as polyurethane (PU) or polyethylene (PE) combined with silicone gel. However, the PU, PE and other plastic materials cannot withstand high temperatures, thereby preventing the existing silicone gel cosmetic patch from being sterilized by high temperatures or boiling. As a result, the silicone gel cosmetic patch is not reusable, causing a waste of resources. Therefore, it is necessary to improve the existing silicone gel cosmetic patches.

### SUMMARY

In view of the defects in the prior art, the present disclosure aims to provide a reusable silicone gel cosmetic patch that can effectively solve the problem of conventional silicone gel cosmetic patches being non-reusable.

In order to achieve the foregoing objectives, the present disclosure adopts the following technical solution:
a reusable silicone gel cosmetic patch, including a silicone base layer, a textile fabric layer, and a silicone gel layer, where the silicone base layer, the textile fabric layer, and the silicone gel layer are integrally formed, and the textile fabric layer is sandwiched between the silicone base layer and the silicone gel layer.

Preferably, the silicone gel layer and the silicone base layer are cross-linked and bonded together through the textile fabric layer to form an integrated structure.

Preferably, the silicone base layer is made of an environmentally-friendly silicone material.

Preferably, the textile fabric layer is made of cotton or linen.

Preferably, the silicone gel layer is made of a self-adhesive and environmentally-friendly silicone material.

Compared with the prior art, the present disclosure has obvious advantages and beneficial effects, as demonstrated by the following technical solution: the cosmetic patch provided by the present disclosure is integrally formed by a silicone base layer, a textile fabric layer, and a silicone gel layer, and the textile fabric layer is sandwiched between the silicone base layer and the silicone gel layer. Since the textile fabric layer can withstand high temperatures, the cosmetic patch can be sterilized by high temperatures or boiling, making it suitable for repeated use. In addition, due to the traction of the textile fabric layer, the cosmetic patch can be repeatedly applied and removed without deformation, thereby effectively solving the problem of resource waste. Therefore, the cosmetic patch provided by the present disclosure is a safer, more environmentally friendly, and practical cosmetic auxiliary product suitable for widespread application.

### BRIEF DESCRIPTION OF THE DRAWINGS

FIG. 1 is a perspective schematic view of assembly according to a preferred embodiment of the present disclosure.

FIG. 2 is an exploded view according to a preferred embodiment of the present disclosure.

FIG. 3 is a cross-sectional view according to a preferred embodiment of the present disclosure.

Reference numerals in the accompanying drawings: 10. silicone base layer; 20. textile fabric layer; and 30. silicone gel layer.

### DETAILED DESCRIPTIONS OF THE EMBODIMENTS

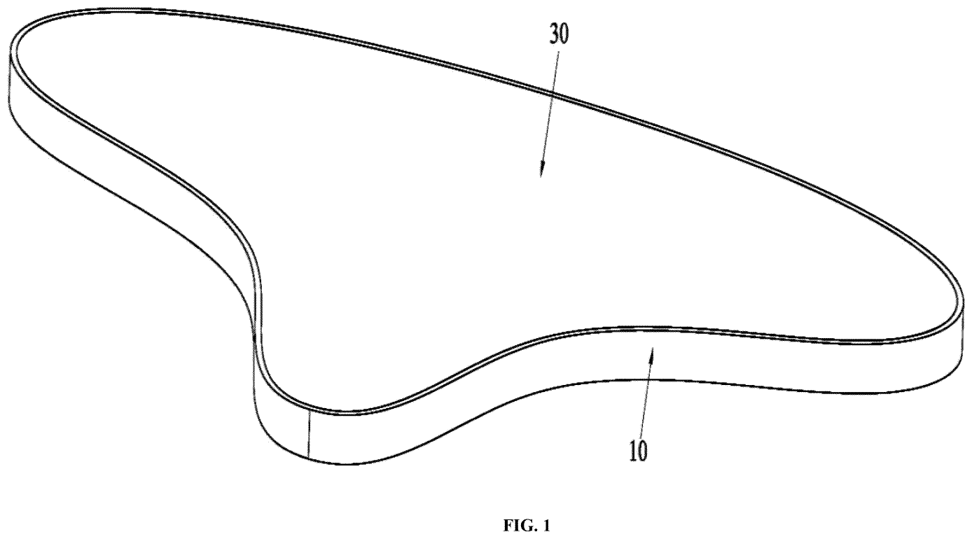

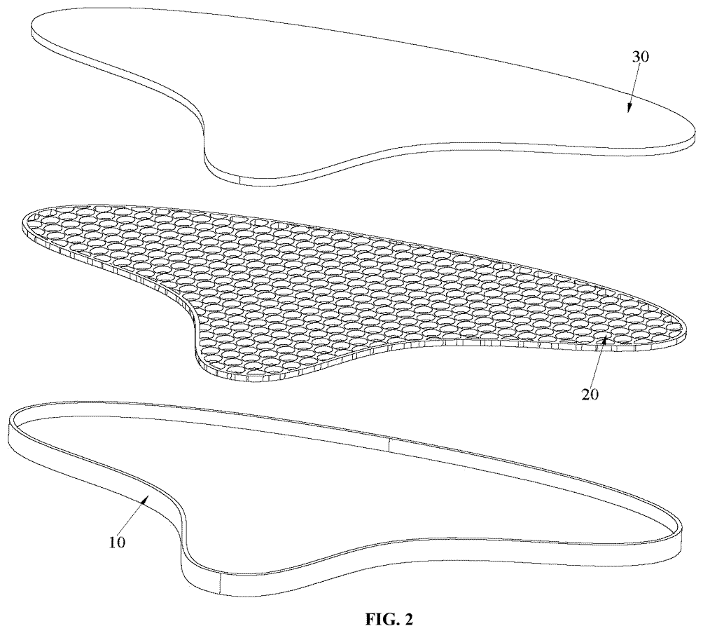

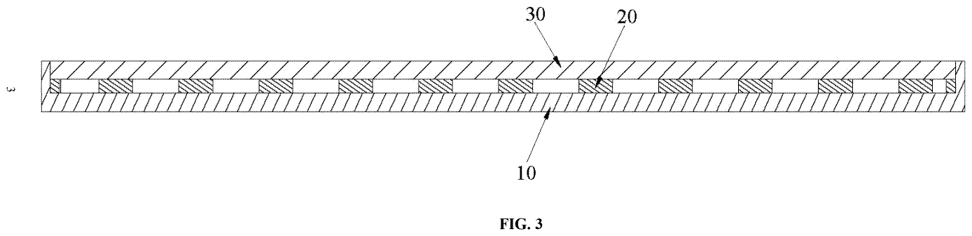

Referring to FIGS. 1-3, a specific structure according to a preferred embodiment of the present disclosure is provided, where the structure includes a silicone base layer 10, a textile fabric layer 20, and a silicone gel layer 30.

The silicone base layer 10, the textile fabric layer 20, and the silicone gel layer 30 are laminated and fixed together in sequence, and the textile fabric layer 20 is sandwiched between the silicone base layer 10 and the silicone gel layer 30. Specifically:
the silicone base layer 10 is an environmentally-friendly and skin-friendly material, and does not contain harmful substances such as bisphenol A and plasticizers. The silicone base layer 10 is made of an environmentally-friendly silicone material. The textile fabric layer 20 is made of cotton or linen. The silicone gel layer 30 exhibits softness, strong adhesiveness, painless removal from the skin, and the ability to be repeatedly applied and removed, and the silicone gel layer 30 is made of a self-adhesive and environmentally-friendly silicone material. Further, the silicone gel layer 30 and the silicone base layer 10 are cross-linked and bonded together through the textile fabric layer 20 to form an integrated structure, such that an overall structural connection of the product is more firm and reliable. Due to the traction of the textile fabric layer 20, the cosmetic patch can be repeatedly applied and removed without deformation, thereby solving the problem of resource waste.

When in use, a cosmetic product such as essence or moisturizing lotion is applied to a surface of the silicone gel layer 30, and the silicone gel layer 30 is then adhered to a surface of a human skin for cosmetic purposes. After use, the cosmetic patch may be sterilized by high temperatures or boiling for subsequent repeated reuse. The cosmetic patch is safer, more environmentally friendly, and practical.

The key design focus of the present disclosure lies in the integrated structure formed by the silicone base layer, the textile fabric layer, and the silicone gel layer, the textile fabric layer is sandwiched between the silicone base layer and the silicone gel layer. Since the textile fabric layer can withstand high temperatures, the cosmetic patch can be sterilized by high temperatures or boiling, making it suitable for repeated use. In addition, due to the traction of the textile fabric layer, the cosmetic patch can be repeatedly applied and removed without deformation, thereby effectively solving the problem of resource waste. Therefore, the cosmetic patch provided by the present disclosure is a safer, more environmentally friendly, and practical cosmetic auxiliary product and is worthy of promotion

The technical principle of the present disclosure is described above in combination with specific embodiments. The description is only for the purpose of illustrating the principle of the present disclosure and is not to be construed in any way as limiting the protection scope of the present disclosure. On the basis of explanations herein, those skilled in the art can devise other specific implementations of the present disclosure without inventive efforts, which fall within the protection scope of the present disclosure.

What is claimed is:

1. A reusable silicone gel cosmetic patch, comprising a silicone base layer, a textile fabric layer, and a silicone gel layer, wherein the silicone base layer, the textile fabric layer, and the silicone gel layer are integrally formed, and the textile fabric layer is sandwiched between the silicone base layer and the silicone gel layer.

2. The reusable silicone gel cosmetic patch according to claim 1, wherein the silicone gel layer and the silicone base layer are cross-linked and bonded together through the textile fabric layer to form an integrated structure.

3. The reusable silicone gel cosmetic patch according to claim 1, wherein the silicone base layer is made of an environmentally-friendly silicone material.

4. The reusable silicone gel cosmetic patch according to claim 1, wherein the textile fabric layer is made of cotton or linen.

5. The reusable silicone gel cosmetic patch according to claim 1, wherein the silicone gel layer is made of a self-adhesive and environmentally-friendly silicone material.
